SEQUENCE LISTING (7 sequences)

SEQ ID NO: 1 - protein, 14 aa, Artificial Sequence
  PEST Motif
SPAETPESPPATPK

SEQ ID NO: 2 - DNA, 24 bp, Artificial Sequence
  Probe 1
ttgcctctcggaatgcatcgctca

SEQ ID NO: 3 - DNA, 20 bp, Artificial Sequence
  Forward Primer 1
gtattcgtcagccgtaagtc

SEQ ID NO: 4 - DNA, 20 bp, Artificial Sequence
  Reverse Primer 1
gcgtcagccaagttaatcag

SEQ ID NO: 5 - DNA, 22 bp, Artificial Sequence
  Probe 2
tgctggcaccagacttgccctc

SEQ ID NO: 6 - DNA, 20 bp, Artificial Sequence
  Forward Primer 2
cggctaccacatccaaggaa

SEQ ID NO: 7 - DNA, 18 bp, Artificial Sequence
  Reverse Primer 2
gctggaattaccgcggct

(19) United States
(12) Patent Application Publication
(10) Pub. No.: US 2011/0081317 A1
(43) Pub. Date: Apr. 7, 2011

(54) Enhancing Gene Transfer

(75) Inventors: Stephen Dewhurst, Rochester, NY (US); Christine N. Zanghi, Rochester, NY (US); Ketna Volcy, Rochester, NY (US); Jacob J. Schlesinger, Pittsford, NY (US)

(73) Assignee: UNIVERSITY OF ROCHESTER, Rochester, NY (US)

(21) Appl. No.: 12/672,386

(22) PCT Filed: Aug. 8, 2008
(86) PCT No.: PCT/US08/72574

§ 371 Date: Feb. 5, 2010

Related U.S. Application Data
(60) Provisional application No. 60/954,631, filed on Aug. 8, 2007.

(51) Int. Cl. A61K 35/76; C12N 5/071; A61K 31/7088; C12N 15/63; A61P 37/04

(52) U.S. Cl. 424/93.2; 435/325; 514/44 R; 435/320.1

(57) ABSTRACT

Described herein are methods of improving the efficiency of gene transfer for a wide range of applications. Specifically provided are methods of increasing expression of an exogenous gene in a cell by contacting the cell with a vector comprising the exogenous gene and contacting the cell with a proteasome inhibitor, a lysosomal protease inhibitor and/or a microtubule inhibitor. Also provided are methods of delivering an antigen delivery vector to a cell or a subject. Provided are antigen delivery systems and kits comprising an antigen delivery vector and a proteasome inhibitor, a lysosomal protease and/or a microtuhulc inhibitor.

### CROSS-REFERENCE TO RELATED APPLICATIONS

This application claims priority to U.S. Provisional Application No. 60/954,631 filed Aug. 8, 2007, which is incorporated herein by reference in its entirety.

### STATEMENT REGARDING FEDERALLY FUNDED RESEARCH

This invention was made with government support under Grant No. R21 A1058791 awarded by the National Institutes of Health (NIH). The government has certain rights in the invention.

### BACKGROUND

The use of phage in protein or antigen delivery has been proposed, but the development of phage-based vaccines has centered on phage display of antigenic peptides linked to filamentous (M13) coat proteins. These vaccines have successfully induced antibody and some cytolytic responses in laboratory animals, but the T-cell response is often weaker than those observed in mammalian viral vectors. Furthermore, these approaches are limited to short antigenic epitopes, due to the constraints on surface display of peptides on filamentous phage, and they do not permit new antigen synthesis in mammalian cells because the surface-modified phage lack a mammalian expression cassette.

Viral vectors are also used for protein delivery. The majority of animal viruses enter the cell through the endo-lysosomal pathway. Some viruses have adopted different mechanisms to escape the endosome or lysosome to enter the cellular cytosol and, if necessary, to travel to the nucleus for gene expression. However, the endosome and lysosome contribute to the inefficient transduction of cells by viral vectors.

### SUMMARY

Described herein are methods of improving the efficiency of gene transfer for a wide range of applications. Specifically provided are methods of increasing expression of an exogenous gene in a cell by contacting the cell with a vector comprising the exogenous gene and contacting the cell with a proteasome inhibitor, a lysosomal protease inhibitor and/or a microtubule inhibitor. For example, the methods comprise contacting the cell with a bacteriophage (e.g., bacteriophage lambda) or viral vector having the exogenous gene and contacting the cell with the inhibitor (e.g., lactacystin, bortezomib, cathepsin B or L inhibitor, or nocodazole). Also provided are methods of delivering an antigen delivery vector to a cell or a subject. The methods include contacting the cell or administering to the subject an antigen delivery vector that includes a bacteriophage or viral vector encoding the antigen and a proteasome inhibitor, a lysosomal protease inhibitor and/or a microtubule inhibitor.

Provided are antigen delivery systems comprising an antigen delivery vector and a proteasome inhibitor, a lysosomal protease inhibitor and/or a microtubule inhibitor.

Kits comprising an antigen delivery vector and a proteasome inhibitor, a lysosomal protease inhibitor and/or a microtubule inhibitor are also provided.

### DESCRIPTION OF DRAWINGS

FIG. 1A is a graph showing wild-type phage mediated luciferase gene expression is enhanced by proteasome inhibitors in HEK 293 cells. FIG. 1B is a graph showing similar data using a phage with a modified coat protein. Luciferase-encoding lambda phage particles were generated, displaying either a wild-type major coat protein, gpD (WT) and a recombinant form of gpD, bearing a PEST motif (SPAETPESPPATPK (SEQ ID NO: 1; phage particles displaying this peptide are hereafter designated “Tpell”). Phage particles were then added to HEK 293 cells at a multiplicity of infection (MOI) of 1×10⁶, and cells were incubated in the presence of the proteasome inhibitors, lactacystin (3 μM), bortezomib (10 nM) or MG132 (1 μM). Sixteen (16) hours later, the cells were washed, and the culture medium was replaced with medium that did not contain proteasome inhibitors. Forty-eight (48) hours following addition of phage, the cells were harvested and lysed, and luciferase activity was measured. Exposure of cells to the various proteasome inhibitors resulted in a profound increase in phage-mediated luciferase gene expression. This result achieved statistical significance for both WT and Tpell phage, indicated by the asterisk, in the case of MG132 (one way ANOVA; p value<0.05, when comparing MG132 treated cells to the untreated control cells); a strong trend is also apparent for lactacystin and bortezomib.

FIG. 2 is a graph showing phage-mediated luciferase gene transfer in HEK 293 cells. Luciferase-encoding Tpell phage particles were added to HEK 293 cells at a multiplicity of infection of 1×10⁶, and cells were incubated in the presence of bortezomib (10 nM) for 24 hours. Cells were then washed, and the culture medium was replaced with medium that did not contain proteasome inhibitors. Forty-eight (48) hours following addition of phage, the cells were harvested and lysed, and luciferase activity was measured. Extended exposure of cells to bortezomib resulted in a profound increase in phage-mediated luciferase gene expression as compared to control cells in the absence (- -) of bortezomib. This result achieved statistical significance, indicated by the asterisk (two-tailed paired t test; p value<0.005, when comparing bortezomib treated cells to the untreated control cells).

FIG. 3 is a graph showing luciferase gene transfer efficiency by Tpell phage with a functional PEST motif (wild-type Tpell motif (Tpell-WT)) and Tpell phage lacking the PEST consensus element (Tpell-SA). The phage were generated, and used to transduce HEK 293 cells. Cells were exposed to luciferase-encoding phage particles at a MOI of 1×10⁶. Forty-eight (48) hours following addition of phage, the cells were harvested and lysed, and luciferase activity was measured. Exposure of cells to the Tpell-SA phage resulted in an enhanced efficiency of phage-mediated luciferase gene expression, when compared to Tpell-WT phage (the asterisk denotes p<0.05, when compared to cells exposed to Tpell-WT phage, as determined by two tailed paired t test).

FIG. 4A is a graph showing proteasome inhibitors enhance wild-type phage-mediated gene transfer in COS cells. FIG. 4B is a graph showing proteasome inhibitors enhance Tpell phage-mediated gene transfer in COS cells. Luciferase-encoding lambda phage particles were generated, displaying either a wild-type major coat protein, gpD (WT) or a modified form of gpD (“Tpell”). Phage particles were then added to COS cells at a MOI of 1×10⁶, and cells were incubated in the presence of the proteasome inhibitors, lactacystin (3 μM), bortezomib (10 nM) or MG132 (1 μM) as described in the legend to FIGS. 1A and 1B. Sixteen (16) hours later, the cells were washed, and the culture medium was replaced with medium that did not contain proteasome inhibitors. Forty-eight (48) hours following addition of phage, the cells were harvested and lysed, and luciferase activity was measured. Exposure of cells to the various proteasome inhibitors enhanced phage-mediated luciferase gene expression. This result achieved statistical significance for the Tpell phage, indicated by the asterisk, in the case of bortezomib (one way ANOVA; p value<0.05, when comparing MG132 treated cells to the untreated control cells); a strong trend is also apparent for MG132 and lactacystin.

FIG. 5 is a graph showing luciferase expression using a plasmid vector encoding an identical luciferase expression cassette in the presence of bortezomib (Bort.) or absence (- -) of bortezomib. HEK 293 cells were transiently transfected with a DNA plasmid containing the same combination of luciferase reporter gene and CMV promoter present in the genome of bacteriophage constructs. Cells were transiently transfected with this plasmid DNA using Lipofectamine and were maintained in the presence of bortezomib (10 nM) for 16 hours. Cells were then washed and returned to normal medium. Forty-eight (48) hours following addition of phage, the cells were harvested and lysed, and luciferase activity was measured. Exposure of cells to bortezomib had no effect on plasmid-mediated luciferase gene expression.

FIGS. 6A-6C are graphs showing bafilomycin A fails to enhance phage-mediated luciferase gene transfer, despite effectively raising endosomal pH. HEK 293 (FIG. 6A) or COS (FIG. 6B) cells were incubated with luciferase-encoding Tpell phage at a MOI of 1×10⁶. Bafilomycin A was added to the culture media at the indicated doses. Twenty-four (24) hours later the cells were washed, and placed in medium lacking the endosomotropic drugs. Forty-eight (48) hours following addition of phage, the cells were harvested and lysed, and luciferase activity was measured. Exposure of cells to bafilomycin A had no effect on phage-mediated luciferase gene expression. In FIG. 6C, HEK 293 cells were pulsed with medium containing fluorescein (F) and tetramethylrhodamine (T) dextran (70 kD; Invitrogen, Carlsbad, Calif.) in the presence or absence (- -) of bafilomycin A for 1.5 hours. Cells were washed and fresh media were added with or without (- -) drug. Two (2) hours later, cells were washed, trypsinized and resuspended in fresh media for flow cytometric analysis of F and T fluorescence; the ratio of T (pH-sensitive) to F (pH insensitive) fluorescence was then calculated. A standard curve was generated by treating HEK 293 cells with F,T-dextran as above, in the absence of bafilomycin A, and then resuspending the cells after trypsinization in media at pH 4.0,4.4, 5.0, 5.4, 6.0, 6.4 in the presence of nigericin (2 μg/ml), prior to performing flow cytometric analysis. The ratio of T/F fluorescence was then plotted against pH, to generate a standard curve; data from the bafilomycin-treated and non-treated cells were then extrapolated to this curve, in order to calculate endosomal pH.

FIGS. 7A-7C are graphs showing omeprazole and brefeldin A fail to enhance phage-mediated luciferase gene transfer, whereas high concentrations of chloroquine enhance phage-mediated luciferase gene transfer. In FIGS. 7A and 7B, HEK 293 (FIG. 7A) or COS (FIG. 7B) cells were incubated with luciferase-encoding Tpell phage at a MOI of 1×10⁶. The indicated endosomotropic drugs were added to the culture medium at doses of 50 μM (omeprazole), 500 ng/ml (brefeldin A) and 50 μM or 70 μM (chloroquine) for HEK 293 and COS cells, respectively. Twenty-four (24) hours later the cells were washed, and placed in medium lacking the endosomotropic. Forty-eight (48) hours following addition of phage, the cells were harvested and lysed, and luciferase activity was measured. Exposure of cells to omeprazole or brefeldin A had no effect on phage-mediated luciferase gene expression. In contrast, treatment of cells with high concentrations of chloroquine resulted in a statistically significant enhancement of phage-mediated gene transfer (the asterisk denotes p<0.05, when compared to untreated (−) cells, as determined by one-way ANOVA with Tukey's post-test). In FIG. 7C, HEK 293 cells were incubated with luciferase-encoding Tpell phage at a MOI of 1×10⁶. Chloroquine was added to the culture medium at the indicated doses. Twenty-four (24) hours later the cells were washed, and placed in medium lacking the endosomotropic drugs. Forty-eight (48) hours following addition of phage, the cells were harvested and lysed, and luciferase activity was measured. Exposure of cells to a high dose of chloroquine resulted in a statistically significant enhancement of phage-mediated gene transfer (the asterisk denotes p<0.05, when compared to untreated cells, as determined by one-way ANOVA with Tukey's post-test).

FIGS. 8A and 8B are graphs showing the effect of cathepsin inhibitors on phage-mediated luciferase gene transfer using luciferase-encoding lambda phage particles, displaying either a wild-type major coat protein, gpD (WT) (FIG. 8A) or a recombinant form of gpD bearing a PEST motif (“Tpell”) (FIG. 8B). Phage particles were added to HEK 293 cells, and cells were incubated in the presence of cathepsin B inhibitor (CatB), cathepsin L inhibitor (CatL) or both (CatB+CatL). Exposure of cells to the various cathepsin inhibitors resulted in a increase in phage-mediated luciferase gene expression.

FIG. 9 is a graph showing phage-mediated luciferase gene transfer in the absence (- -) or presence of cathepsin B inhibitor (CatB), cathepsin L inhibitor (CatL), cathepsin B inhibitor plus cathepsin L inhibitor (CatB+L), bortezomib (Bort.), chloroquine (CHQ), or combinations of these agents (e.g., CaB/L+Bort., CHQ+CatB+L). HEK 293 cells were incubated with luciferase-encoding Tpell phage at a MOI of 1×10⁶. Twenty-four (24) hours later the cells were washed, and placed in medium lacking the drugs. Forty-eight (48) hours following addition of phage, the cells were harvested and lysed, and luciferase activity was measured. Exposure of cells to bortezomib alone (Bort.) or to CatB plus CatL inhibitors (CatB+L) resulted in a modest increase in phage-mediated luciferase gene expression, while treatment of cells with a combination of these agents (CatB/L+Bort.) resulted in a synergistic and statistically significant enhancement of phage-mediated gene transfer (the asterisk denotes p<0.001, when compared to untreated cells or cells exposed to either agent alone, as determined by one-way ANOVA with Tukey's post-test). Exposure of cells to a high concentration of chloroquine (CHQ) resulted in a strong and statistically significant increase in phage-mediated gene transfer (p<0.001, when compared to untreated cells; one-way ANOVA with Tukey's post-test). Co-treatment of cells with CHQ in combination with CatB plus CatL inhibitors did not result in any further increase in luciferase expression, compared to cells exposed to CHQ alone.

FIG. 10 is a graph showing phage DNA levels in HEK 293 cells incubated with Tpell phage and a proteasome inhibitor. The cells were incubated with luciferase-encoding Tpell phage at a MOI of 1×10⁶, in the presence (Bort.) or absence (- -) of bortezomib (10 nM). Sixteen (16) hours later the cells were washed, and placed in medium lacking the drug. Twenty-four (24) hours following addition of phage, the cells were harvested, fractionated and lysed. Nuclear phage DNA levels were then quantitated by DNA PCR analysis using a TaqMan® (Roche Molecular Systems, Inc., Pleasanton, Calif.) primer/probe set specific for the lambda phage integrase gene. Phage DNA levels were then normalized to measured levels of cellular DNA (18S rRNA DNA), and are shown as copies of lambda phage genomic DNA per HEK 293 cell. The analysis was performed in triplicate (three separate wells of cells), and results are presented as the mean of these results; the bars represent the standard error of the mean. Treatment of cells with bortezomib resulted in a statistically significant increase in nuclear accumulation of phage DNA (p<0.01, when compared to untreated cells; one-way ANOVA with Tukey's post-test).

FIG. 11 is a graph showing phage-mediated luciferase gene expression in cells treated with a microtubule inhibitor. CV1 cells stably expressing a cellular Fc receptor (CD64) and its associated gamma chain were pretreated with nocodazole (5 μM) or paclitaxel (20 μg/ml), for 30 minutes at 37° C. Preformed lambda phage: antibody complexes (generated by incubating wild-type luciferase-encoding phage particles with gpD-specific rabbit IgG antibodies) were added to the cells. Cells were harvested 48 hours later and lysed and luciferase activity was measured. The data are representative of three independent experiments that yielded similar results. The asterisks (**, ***) denote a statistically significant difference from control cells/conditions (p value<0.05[**] or p value<0.001 [***], one-way ANOVA).

FIG. 12 is a graph showing plasmid mediated luciferase gene transfer in cells treated with a microtubule inhibitor. A DNA plasmid encoding a luciferase reporter gene was mixed with Lipofectamine. This was then added to COS cells that had been stably transfected with expression plasmids encoding a cellular Fc receptor (CD64) and its associated gamma chain, in the presence or absence (DNA only and DNA+DMSO) of latrunculin A (120 nM), paclitaxel (20 μg/ml), or nocodazole (5 μM). Cells were harvested 48 hours later and lysed, and luciferase activity measured.

FIG. 13 is a graph showing, in cells treated with paclitaxel, nocodazole, or latrunculin A, adenoviral-mediated luciferase gene transfer. Latrunculin A (10 nM), paclitaxel (20 μg/ml), or nocodazole (5 μM) was added to cells 30 minutes prior to transduction of COS-7 cells with a luciferase-expressing adenovirus vector (AdLucGFP) at a multiplicity of infection (MOI) of 10. Media were changed 24-hours post-transfection, and cells were lysed in Passive Lysis Buffer 24 hours later. Protein quantities were standardized and luciferase activity was measured in the cell lysates. The mean control level is shown as Ad only.

### DETAILED DESCRIPTION

Vectors such as, for example, bacteriophage, can be used to express foreign genes in mammalian cells and tissues. By way of example, bacteriophage lambda (λ) has certain appealing characteristics as an antigen or antigen delivery vector. Lambda is a dsDNA, temperate phage, 50 nm wide and about 150 nm long; this is a size comparable to most mammalian viruses, including HIV Lambda can accept inserts and genomic deletions anywhere between 78% and 105% of the wild-type genome, allowing for insertion of up to 15 kb. Finally, lambda is extremely stable under multiple storage conditions, including desiccation, and large-scale production of lambda is rapid and relatively inexpensive making it a versatile option for vaccine administration to low income nations. Phage are inexpensive to produce and purify, genetically tractable, and have a substantial track record of safe use in humans and research animals in large quantities for the treatment of bacterial infections.

Presented herein are methods and vectors that will more efficiently transduce mammalian cells, and thereby elicit stronger antigenic responses to encoded antigens, for purposes of eliciting an immune response against various infectious agents and diseases (e.g., cancer, neurologic diseases and other disorders and conditions). Thus, the provided methods, vectors and systems can be used to deliver antigens to a cell or subject in need of vaccination.

As described in the Examples below, the efficiency of gene transfer was increased in the presence of pharmacologic agents that inhibit proteasome function or microtubule formation. Efficiency of gene transfer was also increased in the presence of lysosomal protease inhibitors such as, for example, chloroquine and inhibitors of cathepsin. Chloroquine is also known to inhibit endosome acidification. Thus, provided are methods of enhancement of phage-mediated or viral vector-mediated gene transfer using proteasome inhibitors. Also provided are methods of enhancement of phage-mediated gene transfer using lysosomal protease inhibitors such as, for example, inhibitors of cathepsin and chloroquine. Methods of enhancement of phage-mediated, DNA plasmid-mediated or viral vector-mediated gene transfer using agents that disrupt microtubules are also provided. Specifically, provided are methods of increasing expression of an exogenous gene in a cell, comprising contacting the cell with a bacteriophage or viral vector comprising the exogenous gene and contacting the cell with a proteasome inhibitor. Contacting the cell with the proteasome inhibitor results in an increase in expression of the exogenous gene in the cell as compared to a control. Optionally, the methods further comprises contacting the cell with a lysosomal protease inhibitor and/or a microtubule inhibitor.

Provided are methods of increasing expression of an exogenous gene in a cell, comprising contacting the cell with a bacteriophage, plasmid or viral vector comprising the exogenous gene, and contacting the cell with an agent selected from the group consisting of a proteasome inhibitor, a lysosomal inhibitor and a microtubule inhibitor. Contacting the cell with the agent results in an increase in expression of the exogenous gene in the cell as compared to a control.

Also provided are methods of increasing expression of an exogenous gene in a cell, comprising contacting the cell with a bacteriophage comprising the exogenous gene and contacting the cell with a lysosomal protease inhibitor such as, for example, a cathepsin inhibitor or chloroquine. Contacting the cell with the lysosomal protease inhibitor results in an increase in expression of the exogenous gene in the cell as compared to a control. Optionally, the method further comprises contacting the cell with a proteasome inhibitor and/or a microtubule inhibitor.

Provided are methods of increasing expression of an exogenous gene in a cell. The methods include contacting the cell with a non-viral vector comprising the exogenous gene and contacting the cell with a microtubule inhibitor. Contacting the cell with the microtubule inhibitor results in an increase in expression of the exogenous gene in the cell as compared to a control. The non-viral vector can be a plasmid or a bacteriophage. Thus, provided are methods of increasing expression of an exogenous gene in a cell comprising contacting the cell with a bacteriophage with the exogenous gene and contacting the cell with a microtubule inhibitor. Contacting the cell with the microtubule inhibitor results in an increase in expression of the exogenous gene in the cell as compared to a control.

Methods of delivering an antigen delivery vector to a cell are provided. The methods include the steps of contacting the cell with an antigen delivery vector, wherein the antigen delivery vector encodes an antigen and contacting the cell with an agent selected from the group consisting of a proteasome inhibitor, lysosomal protease inhibitor such as, for example, a cathepsin inhibitor and chloroquine, a microtubule inhibitor, or a combination thereof. Combinations of the agents can also be used in the methods herein. Also provided are methods of delivering an antigen delivery vector to a subject. The methods comprise the steps of administering to the subject an antigen delivery vector, wherein the antigen delivery vector encodes an antigen and administering to the subject an agent selected from the group consisting of a proteasome inhibitor, a lysosomal protease inhibitor such as, for example, a cathepsin inhibitor and chloroquine, and a microtubule inhibitor. Administration of the agent results in an increase expression of the antigen from the antigen delivery vector as compared to a control. If the agent is a proteasome inhibitor or a lysosomal protease inhibitor the antigen delivery vector can be a bacteriophage or viral vector. If the agent is a microtubule inhibitor, the antigen delivery vector can be a plasmid, bacteriophage or viral vector.

The provided methods optionally further comprise contacting a cell with or administering to a subject an immunostimulatory molecule, such as, for example, interferons, cytokines, chemokines and soluble ligands for CD40 receptor. Such molecules and their methods of administration are known:

As used throughout, higher, increases, enhances or elevates as compared to a control refer to increases above a control. For example, a control level can be the level of expression or activity in the same cell or subject prior to or after recovery from a stimulus, or the control level can be the level in a control cell or subject or population of cells or subjects in the absence of a stimulus.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. The subject can be a mammal such as, for example, a primate, such as a human.

Also provided are antigen delivery systems comprising a proteasome inhibitor and an antigen delivery vector encoding an antigen. As used herein, a delivery system or delivery vector facilitates, permits, and/or enhances delivery to a particular site and/or with respect to particular timing. Provided are antigen delivery systems comprising a lysosomal protease inhibitor, such as, for example, a cathepsin inhibitor or chloroquine, and an antigen delivery vector encoding an antigen. Provided are also antigen delivery systems comprising a microtubule inhibitor and an antigen delivery vector encoding an antigen. As used herein, antigen delivery system refers to a composition, wherein the composition can deliver an antigen to antigen presenting cells of a subject for the purpose of eliciting an antigenic response in the subject.

As used herein antigen refers to any substance that stimulates the production of antibodies or expansion of specific T cell clone(s). The term immunogen refers to any substance or organism that provokes an immune response when introduced into the body. It is understood that an antigen can also be an immunogen and vice versa. As used herein antigen presenting cell refers to a cell that carries on its surface antigen bound to MHC Class I or Class II molecules and presents the antigen in this context to T-cells. This can include macrophages, endothelium, dendritic cells and Langerhans cells of the skin, as well as other cell types under certain circumstances. As disclosed and described herein, the provided antigen delivery systems can be used to elicit humoral immunity or cellular immunity.

An antigen is recognized and bound specifically by an antibody or by a T cell antigen receptor. Antigens can include peptides, proteins, glycoproteins, polysaccharides (e.g., Hemophilus influenza antigens), complex carbohydrates, sugars, gangliosides, lipids (e.g., sterols, fatty acids and phospholipids); portions thereof and combinations thereof. Antigens include any molecule capable of eliciting a B cell or T cell antigen-specific response. Preferably, antigens elicit an antibody response specific for the antigen. Optionally, the antigen can be an allergen. The antigen can be from an infectious agent, including protozoan, bacterial, fungal (including unicellular and multicellular), and viral infectious agents. Examples of suitable viral antigens are known. Bacteria include Hemophilus influenza, Mycobacterium tuberculosis and Bordetella pertussis. Protozoan infectious agents include malarial plasmodia, Leishmania species, Trypanosoma species and Schistosoma species. Fungi include Candida albicans. Viral polypeptide antigens include, but are not limited to, HIV proteins, such as HIV gag proteins and HIV polymerase; influenza proteins, such as matrix (M) protein and nucleocapsid (NP) protein; hepatitis B proteins, such as surface antigen (HBsAg), hepatitis B core protein (HBcAg), hepatitis e protein (HBeAg), hepatitis B DNA polymerase, and hepatitis C antigens; and the like. Other examples of antigen polypeptides are group- or sub-group-specific antigens, which are known for a number of infectious agents, including, but not limited to, adenovirus, herpes simplex virus, papilloma virus, respiratory syncytial virus and poxviruses.

Many antigenic peptides and proteins are known, and available in the art; others can be identified using conventional techniques. For immunization against tumor formation or treatment of existing tumors, antigenic agents can include tumor cells (live or irradiated), tumor cell extracts, or protein subunits of tumor antigens such as Her-2/neu, Mart1, carcinoembryonic antigen (CEA), gangliosides, human milk fat globule (HMFG), mucin (MUCI), MAGE antigens, BAGE antigens, GAGE antigens, gp100, prostatic acid phosphatase (PAP), prostate specific antigen (PSA), prostate stem cell antigen (PSCA), and tyrosinase.

Kits and compositions comprising the provided antigen delivery systems are described. Specifically, described are kits comprising an antigen delivery vector and an agent, wherein the agent is a proteasome inhibitor, a lysosomal protease inhibitor such as, for example, a cathepsin inhibitor or chloroquine, or a microtubule inhibitor. In the provided kits, the antigen delivery vector and the agent can be in the same container or in separate containers. Optionally, the provided kits further comprise instructions for use, means for administering one or both of the vector and the agent.

Provided are compositions comprising an antigen delivery vector and an agent, wherein the agent is a proteasome inhibitor, a lysosomal protease inhibitor such as, for example, a cathepsin inhibitor or chloroquine, or a microtubule inhibitor.

The provided kits, compositions and systems can further comprise an immunostimulatory molecule selected from the group consisting of interferons, cytokines, chemokines and soluble ligands for CD40 receptor.

As used throughout, the term antigen delivery vector refers to a vector, such as, for example, a plasmid, viral vector or bacteriophage, that can be used to deliver an antigen to a cell or subject. The viral vector, plasmid, or bacteriophage comprises a nucleic acid that encodes an antigen. The phrase bacteriophage antigen delivery vector as used herein refers to bacteriophage comprising an exogenous gene of interest such as, for example, an antigen. The phage of the provided delivery vectors can comprise a surface polypeptide modified to target a selected cell (e.g., antigen-presenting cells). As used herein, modified refers to any alteration(s) (including, for example, genetic alterations) that affects either form or function. For example, the modifications to phage vectors provided herein include modifications designed to increase phage survival in the human host and/or to enhance phage binding to mammalian cells. As used herein, surface polypeptide refers to a native or heterologous polypeptide that is expressed by and exposed on the phage surface. It is understood that a molecule can be displayed on the surface of the phage by conjugating the molecule to a surface polypeptide. For example, bacteriophage lambda can be modified to display PEST-like motifs on the surface of the bacteriophage. Phage vectors and methods for making and using phage vectors are described in WO 2007101,5704, which is incorporated by reference herein in its entirety at least for phage vectors and methods of making and using phage vectors including lambda phage vectors modified to display PEST-like motifs on the surface of the phage.

The vectors comprising nucleic acids encoding one or more polypeptides provided herein, for example, an antigen, can be operably linked to an expression control sequence. Preferred promoters controlling transcription from vectors in mammalian host cells may be obtained from various sources, for example, the genomes of viruses such as polyoina, Simian Virus 40 (SV40), adenovirus, retroviruses, hepatitis B virus and most preferably cytomegalovirus, or from heterologous mammalian promoters (e.g., beta actin promoter or EF1 promoter). The promoter can be a hybrid or chimeric promoter (e.g., a cytomegalovirus promoter fused to the beta actin promoter). The early and late promoters of the SV40 virus are conveniently obtained as an SV40 restriction fragment that also contains the SV40 viral origin of replication. The immediate early promoter of the human cytomegalovirus is conveniently obtained as a Hind111 E restriction fragment. Promoters from the host cell or subject or related species also are useful herein.

Vectors provided herein optionally contain an enhancer. Enhancer generally refers to a sequence of DNA that functions at no fixed distance from the transcription start site and can be either 5′ or 3′ to the transcription unit. Enhancers can be within an intron as well as within the coding sequence itself. They are usually between 10 and 300 base pairs in length, and they function in cis. Enhancers usually function to increase transcription from nearby promoters. Enhancers can also contain response elements that mediate the regulation of transcription. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone, synthetic transcription factors, directed RNA self-cleavage and other approaches known to those of skill in the art. There are also ways to enhance viral vector gene expression by exposure to irradiation, such as gamma irradiation, or alkylating chemotherapy drugs.

The promoter and/or enhancer can act as a constitutive promoter and/or enhancer to maximize expression of the region of the transcription unit to be transcribed. A promoter of this type is the CMV promoter (650 bases). Other promoters are SV40 promoters, cytomegalovirus (plus a linked intron sequence), beta-actin, elongation factor-1 (EF-1) and retroviral vector LTR.

Vectors may also contain sequences necessary for the termination of transcription which may affect mRNA expression. These regions are transcribed as polyadenylated segments in the untranslated portion of the mRNA. The 3′ untranslated regions also include transcription termination sites. The identification and use of polyadenylation signals in expression constructs is well established. In certain transcription units, the polyadenylation region is derived from the SV40 early polyadenylation signal and consists of about 400 bases.

The terms peptide, polypeptide, protein or peptide portion are used broadly herein to mean two or more amino acids linked by a peptide bond and are not used herein to suggest a particular size or number of amino acids comprising the molecule. The term fragment is used herein to refer to a portion of a full-length polypeptide or protein.

By isolated or purified is meant a composition (e.g., a polypeptide or nucleic acid) that is substantially free from other materials with which the composition is normally associated in nature. For example, polypeptides or fragments thereof, can be obtained, for example, by extraction from a natural source (e.g., phage), by expression of a recombinant nucleic acid encoding the polypeptide (e.g., in a cell or in a cell-free translation system), or by chemically synthesizing the polypeptide. In addition, polypeptide fragments may be obtained by any of these methods, or by cleaving full length polypeptides.

Proteasomes are responsible for the selective degradation of proteins when cells no longer need them. Proteasome inhibitors are drugs that block the action of proteasomes, which are cellular complexes that break down proteins. Proteasome inhibitors suitable for use in the provided methods include, but are not limited to, bortezomib (VELCADE® (Millenium Pharmaceuticals, Cambridge, Mass.)), lactacystin, MG132, peptide aldehydes, epoxomicin and derivatives of epigallocatechin-3-gallate (Landis-Piwowar et al., Bioorg. Med. Chem. 15(15:5076-82 (2007)).

Lysosomal proteases are also responsible for degradation of proteins. As shown in the examples below, chloroquine and inhibitors of the major lysosomal proteases, cathepsins B and L, resulted in a strong enhancement of phage-mediated gene transfer. Chloroquine is also known as an endosome acidification inhibitor. Suitable lysosomal proteases for use in the provided methods, compositions and systems include, but are not limited to, a cathepsin inhibitor, chloroquine, antipain hydrochloride, chymostatin, trans-eposycuccinyl-1-leucylamido-(4-guanidino)butanc, leupeptin, pepstatin A, CA074Me, ZPAD, serpinB3, and cystatin C.

Microtubules are components of the cytoskeleton and are involved in,many cellular processes including mitosis, cytokinesis, and vesicular transport. Microtubule dynamics can be altered by drugs. For example, the taxane drug class (e.g., paclitaxel or docetaxel), used in the treatment of cancer, blocks dynamic instability by stabilizing GDP-bound tubulin in the microtubule. Thus, even when hydrolysis of GTP reaches the tip of the microtubule, there is no depolynerization and the microtubule does not shrink back. Nocodazole and colchicine have the opposite effect, blocking the polymerization of tubulin into microtubules. Microtubule inhibitors suitable for use in the provided methods include, but are not limited to, nocadozole, paclitaxel, vinblastine, vincristine, colchicine, vinorelbine, vindesine, docetaxel, ixabepilone, SB-7 15992, SB-74392 1, tryprostatin A, dolastatin 15, podophyllotoxin, and rhzoxin.

The provided agents, vectors, systems and any combination thereof can be formulated into pharmaceutical compositions. Thus the herein provided agents, vectors and systems can be administered in vitro or in vivo in a pharmaceutically acceptable carrier. By pharmaceutically acceptable is meant a material that is not biologically or otherwise undesirable, i.e., the material may be administered to a subject, along with the vector, without causing undesirable biological effects or interacting in a deleterious manner with the other components of the pharmaceutical composition in which it is contained. The carrier is selected to minimize any degradation of the active ingredient and to minimize any adverse side effects in the subject.

The compositions may be administered orally, parenterally (e.g., intravenously), by intramuscular injection, by intraperitoneal injection, intradermally, transdermally, extracorporeally, topically or the like, including topical intranasal administration or administration by inhalant. For example, provided is a method of eliciting an immune response in a subject, comprising intradermally administering to the subject an antigen delivery system provided herein. It has also been shown that lambda is capable of withstanding the harsh conditions encountered during oral administration (Jepson and March (2004) Vaccine 22:2413-19). Orally administered phage have been reported to reach the bloodstream for multiple species of bacteriophage (Hildebrand, and Wolochow (1962) Proc Soc Exp Biol Med 109:183-85; Reynaud et al. (1992) Vet Microbiol 30:203-12; Weber-Dabrowska et al. (1987) Arch Immunol Ther Exp 35:563-68). Furthermore, the specific targeting peptide sequences that allow phage to pass through the intestinal wall and thereby enter the general circulation can be used (Duerr et al. (2004) J Virol Methods 11 6: 177-80).

Pharmaceutical compositions may include carriers, thickeners, diluents, buffers, preservatives and surface active agents in addition to the molecule of choice. Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (21st edition) Lippincott Williams & Wilkins, Philadelphia, Pa. 2005. Typically, an appropriate amount of a pharmaceutically-acceptable salt is used in the formulation to render the formulation isotonic. Examples of a pharmaceutically-acceptable carriers include, but are not limited to, saline, Ringer's solution and dextrose solution. Further carriers may include sustained release preparations such as semipermeable matrices of solid hydrophobic polymers containing the antibody, which matrices are in the form of shaped articles, e.g., films, liposomes or microparticles. It will be apparent to those skilled in the art that certain carriers may be more preferable depending upon, for instance, the route of administration and concentration of composition being administered.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

As used herein, topical intranasal administration means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Formulations for topical administration may include ointments, lotions, creams, gels, drops, sprays, liquids, patches and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Generally phage particles, agents, or antigen delivery systems or vectors are transferred to a biologically compatible solution or pharmaceutically acceptable delivery vehicle, such as sterile saline, or other aqueous or non-aqueous isotonic sterile injection solutions or suspensions, numerous examples of which are well known in the art, including Ringer's, phosphate buffered saline, or other similar vehicles.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein for the methods taught therein.

Pharmaceutical compositions may also include adjuvants or immunostimulants. The adjuvant and/or immunostimulant can be administered concomitantly with, immediately prior to, or after administration of a composition, agent or vector provided herein. Pharmaceutical compositions may also include one or more active ingredients such as antimicrobial agents, anti-inflammatory agents and anesthetics.

Immunostimulants can be selected from the group including, but not limited to, cytokines, chemokines, growth factors, angiogenic factors, apoptosis inhibitors, and combinations thereof. Cytokines may be selected from the group including, but not limited to, interleukins including IL-1, IL-3, IL-2, IL-5, IL-6,IL-12, IL-15 and IL-18; transforming growth factor-beta (TGF-β); granulocyte macrophage colony stimulating factor (GM-CSF); interferon-gamma (IFN-γ); or any other cytokine that has immunostimulant activity. Portions of cytokines, or mutants or mimics of cytokines (or combinations thereof) can also be used in the provided compositions and methods.

Chemokines may optionally be selected from a group including, but not limited to, Lymphotactin, RANTES, LARC, PARC, MDC, TAR C, SLC and FKN. Apoptosis inhibitors may optionally be selected from the group including, but not limited to, inhibitors of caspase-8, and combinations thereof. Angiogenic factors may optionally be selected from the group including, but not limited to, a basic fibroblast growth factor (FGF), a vascular endothelial growth factor (VEGF), a hyaluronan (HA) fragment, and combinations thereof.

Adjuvant refers to a substance which, when added to an immunogenic agent such as antigen, nonspecifically enhances or potentiates an immune response to the agent in the recipient host upon exposure to the mixture. Adjuvants include metallic salts, such as aluminum salts, and are well known in the art as providing a safe excipient with adjuvant activity. The mechanism of action of these adjuvants are thought to include the formation of an antigen depot such that antigen may stay at the site of injection for up to 3 weeks after administration, and also the formation of antigen/metallic salt complexes which are more easily taken up by antigen presenting cells. In addition to aluminum, other metallic salts have been used to adsorb antigens, including salts of zinc, calcium, cerium, chromium, iron, and berilium. The hydroxide and phosphate salts of aluminum are the most common. Formulations or compositions containing aluminum salts, antigen, and an additional immunostimulant are known in the art. An example of an immunostimulant is 3-de-0-acylated monophosphoryl lipid A (3D-MPL). Other suitable adjuvants include, but are not limited to, alum, TLR agonists, saponin derivatives, Ribi, ASO4, montanide and ISA 51. Suitable TLR agonists include TLR9 agonists such as a CpG oligonucleotides, imiquimod, resiquimod, MPL-A, flagellin and derivatives thereof. Suitable saponin derivatives include QS21 and GPI0100.

Other examples of substantially non-toxic, biologically active adjuvants include hormones, enzymes, growth factors, or biologically active portions thereof. Such hormones, enzymes, growth factors, or biologically active portions thereof can be of human, bovine, porcine, ovine, canine, feline, equine, or avian origin, for example, and can be tumor necrosis factor (TNF), prolactin, epidermal growth factor (EGF), granulocyte colony stimulating factor (GCSF), insulin-like growth factor (IGF-1), somatotropin (growth hormone) or insulin, or any other hormone or growth factor whose receptor is expressed on cells of the immune system.

Adjuvants also include bacterial toxins, e.g., the cholera toxin (CT), the heat-labile toxin (LT), the Clostridium difficile toxin A and the pertussis toxin (PT), or combinations, subunits, toxoids, chimera, or mutants thereof. For example, a purified preparation of native cholera toxin subunit B (CTB) can be used. Fragments, homologs, derivatives, and fusions to any of these toxins are also suitable, provided that they retain adjuvant activity. Suitable mutants or variants of adjuvants are described, e.g., in WO 95/17211 (Arg-7-Lys (CT mutant)), WO 96/6627 (Arg-192-Gly (LT mutant)), and WO 95/34323 (Arg-9-Lys and Glu-129-Gly (PT mutant)). Additional LT mutants that can be used in the methods and compositions include, e.g., Ser-63-Lys, Ala-69-Gly,Glu-110-Asp, and Glu-112-Asp mutants. Other adjuvants, such as RH3-ligand; CpG-motif oligonucleotide; a bacterial monophosphoryl lipid A (MPLA) of, e.g., E. coli, Salmonella minnesota, Salmonella typhimurium, or Shigella exseri; saponins (e. g., QS21), or polylactide glycolide (PLGA) microspheres, can also be used. Possible other adjuvants are defensins and CpG motifs.

As used herein, an effective dosage, effective amount or a sufficient amount of a substance is that amount sufficient to effect beneficial or desired results, including clinical results, and, as such, an effective amount depends upon the context in which it is being applied. In the context of administering a composition that modulates an immune response to an antigen, an effective amount is an amount sufficient to achieve such a modulation as compared to the immune response obtained when the antigen is administered alone. An effective amount can be administered in one or more administrations.

Effective dosages of phage depends on a variety of factors and may thus vary somewhat from subject to subject. Effective dosages and schedules for administering the compositions are determined empirically, and making such determinations is within the skill in the art. The exact amount required varies from subject to subject, depending on the species, age, weight and general condition of the subject, the severity of the disease being treated, the particular virus or vector used and its mode of administration. Thus, it is not possible to specify an exact amount for every composition. However, an appropriate amount can be determined by one of ordinary skill in the art using only routine experimentation given the guidance provided herein.

The dosage ranges for the administration of the compositions are those large enough to produce the desired effect in which the symptoms of the disease are affected. The dosage should not be so large as to cause unnecessary adverse side effects, such as unwanted cross-reactions and anaphylactic reactions. The dosage can be adjusted by the individual physician in the event of any counter indications. Dosage can vary, and can be administered in one or more dose administrations daily, for one or several days or can be administered within days, weeks, months or years between administrations.

Following administration of a disclosed composition the efficacy of the composition can be assessed in various ways well known to the skilled practitioner. For instance, one of ordinary skill in the art will understand that a composition disclosed herein is efficacious in eliciting an immune response in a subject by observing a humoral response. For example, the immune response to phage particles at either high or low density can be assessed in animals.

As used in the specification and the appended claims, the singular forms a, an and the include plural referents unless the context clearly dictates otherwise. Thus, for example, reference to a pharmaceutical carrier includes mixtures of two or more such carriers, and the like.

Optional or optionally means that the subsequently described event or circumstance may or may not occur, and that the description includes instances where said event or circumstance occurs and instances where it does not.

Publications cited herein and the material for which they are cited are hereby specifically incorporated by reference in their entireties.

It should be understood that various modifications of the vectors, compositions, antigen delivery systems and methods may be made. Furthermore, when one characteristic or step is described, it can be combined with any other characteristic or step herein even if the combination is not explicitly stated. Accordingly, other vectors, compositions, antigen delivery systems and methods are within the scope of the claims.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope. Efforts have been made to ensure accuracy with respect to numbers (e.g., amounts, temperature, etc.), but some errors and deviations should be accounted for.

### EXAMPLES

### Example 1

### Proteasome Inhibitors Enhance Bacteriophage Lambda (k) Mediated Gene Transfer in Mammalian Cells

Materials and Methods

Preparation of bacteriophage lambda lysogens and vector production. The λD1180(luc) lysogen (Dam15 del EcoRI-Sac1 clts857 nin5 Sam100) has been described (Eguchi et al., J. Biol. Chem. 276(28):26204-10, 2001). The λD1180(luc) phage contains a firefly luciferase reporter gene under the regulatory control of the human cytomegalovirus immediate-early promoter. λD1180(luc) phage particles were prepared from E. coli lysogens that were stably transformed with either a plasmid encoding wildtype gpD or plasmids encoding gpD fusion proteins of interest, as described (Zanghi et al., Nucleic Acids Res. 33(18):e160, 2005). λ(luc) particles were purified by CsCl density gradient centrifugation and titered on LE392 E. coli cells (Zanghi et al., Nucleic Acids Res. 33(18):e160,2005).

Cells lines and phage transduction. Human embryonic kidney (HEK) 293 cells and COS simian kidney cells were obtained from American Type Culture Collection (ATCC), P.O. Box 1549, Manassas, Va. 20108. Cells were cultured in Dulbecco's Modified Eagle's Medium (DMEM) containing 10% fetal bovine serum, plus 2 mM L-glutamine and 100 U/ml penicillin, 100 μg/ml streptomycin. Twenty-four to forty-eight (24-48) hours following addition of phage to cells, cultures were washed with phosphate buffered saline (PBS) and lysed in passive lysis buffer (Promega Corporation, Madison, Wis.). Protein content in the lysates was quantitated by Bradford assay, and equal amounts of lysate (normalized in terms of protein content) were used in luciferase assays. Luciferase assay data are reported in relative light units.

Transient transfection of cells with plasmid DNA. 1×10⁴ HEK 293 cells were seeded into 96-well plates and incubated overnight. Cells were then transfected with 50 ng of a mammalian expression plasmid encoding the luciferase reporter gene (pgWiz-CMV luciferase) using lipofectamine-2000 reagent in the presence or absence of lOnM bortezomib. Four (4) hours thereafter, media were removed and fresh media were added (with or without bortezomib). Cells were incubated for an additional 12 hours, and media were again replaced (this time without any exogenous drug). Forty-eight (48) hours after transfection of the cells, they were washed with PBS, incubated in 1× passive lysis buffer (Promega Corporation, Madison, Wis.) and lyzed using two freeze-thaw cycles. Protein content within the lysates was then quantitated using Bradford assay and equal amounts of lysate (normalized in terms of protein content) were used in luciferase assays. Luciferase assay data are reported in relative light units.

Measurement of endosomal pH. 5×10⁵ HEK293 cells were preincubated in DMEM medium with 10% FBS (DMEM-10) overnight, after which the medium was replaced by DMEM-10 containing 2 mg/ml 70kD fluorescein and tetramethylrhodamine dextran (Invitrogen, Carlsbad, Calif.) plus either 100 nM or 500 nM bafilomycin A1 (BAF) or 2 mg/mL of nigericin (positive control) or no drug (negative control). One and a half (1.5) hours later, cells were washed in warm PBS and fresh media was added (with or without BAF or nigericin, as appropriate). Two (2) hours thereafter, cells were again washed with warm PBS, detached by trypsinization, washed with fresh media (with or without BAF or nigericin, as appropriate), and pelleted by centrifugation (5 minutes, 1000 g at 4° C.). Fluorescein

(F) and tetramethylrhodamine (T) fluorescence were quantitated by flow cytometric analysis using a FACS Calibur (Becton Dickinson). A standard curve was generated by suspending aliquots of cells (in the presence of 2 mg/ml of nigericin) in PBS at pH 4, 4.4, 5, 5.4, 6 and 6.4. The ratio of T/F fluorescence was then plotted against pH, to generate a standard curve; data from the bafilomycin-treated and non-treated cells were then extrapolated to this curve, in order to calculate endosomal pH.

Quantitative analysis of intranuclear lambda phage genomic DNA. HEK 293 cells were incubated with phage lambda at a MOI of 10⁶ in the presence or absence of 10 nM bortezomib. Sixteen (16) hours later, media was replaced with bortezomib-free DMEM (10% FBS); twenty-four (24) hours thereafter, cells were washed with cold PBS and non-internalized phage particles were then removed by performing 3 cold acid washes (0.2M CH3COOH, 0.5M NaCl, pH2.5), as described (Lankes et al., J. Appl. Microbiol 102(5):1337-49, 2007). Cells were then washed once more in PBS, trypsinized, pelleted by centrifugation (5 minutes, 1000 g at 4° C.) and washed again in cold PBS prior to suspension in lysis buffer (genomic DNA extraction kit; Qiagen, Valencia, Calif.). The nuclear cell fraction was separated by centrifugation, according to the manufacturer's instructions, and extracted in nuclear lysis buffer in the presence of proteinase K and RNAse A at 50° C. for 1 hour. DNA was then extracted using a 20/G genomic (Qiagen, Valencia, Calif.). Phage genomic DNA in the nuclear lysate was quantitated by DNA qPCR analysis on a BioRad iCycler using a TaqMan® (Roche Molecular Systems, Inc., Pleasanton, Calif.) primer/probe set specific for the lambda phage integrase gene (Probe: FAM-5′-TTGCCTCTCGGAATGCATCGCTCA-3′-TAMRA (SEQ ID NO:2), Forward-5′-GTATTCGTCAGCCGTAAGTC-3′ (SEQ ID NO:3), Reverse-5′-GCGTCAGCCAAGTTAATCAG-3′ (SEQ ID NO:4)). Cellular chromosomal DNA in the nuclear lysate was also quantitated by DNA qPCR analysis using a TaqMan® (Roche Molecular Systems, Inc., Pleasanton, Calif.) primer/probe set specific for the 18S ribosomal RNA gene (Probe: FAM-5′-TGCTGGCACCAGACTTGCCCTC-3′-TAMRA (SEQ ID NO:5), Forward-5′-CGGCTACCACATCCAAGGAA-3′ (SEQ ID NO:6), Reverse-5′-GCTGGAATTACCGCGGCT-3′ (SEQ ID NO:7)). The calculated copy number of nuclear lambda phage DNA was then normalized to the copy number of 18S cellular DNA, and results were expressed as the number of copies of lambda phage DNA per cell.

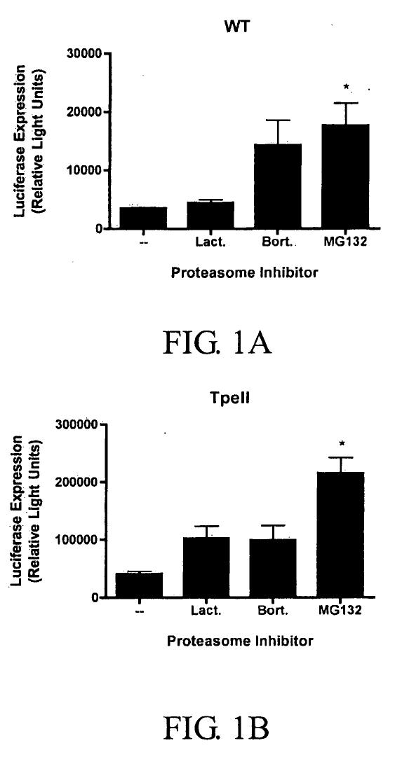

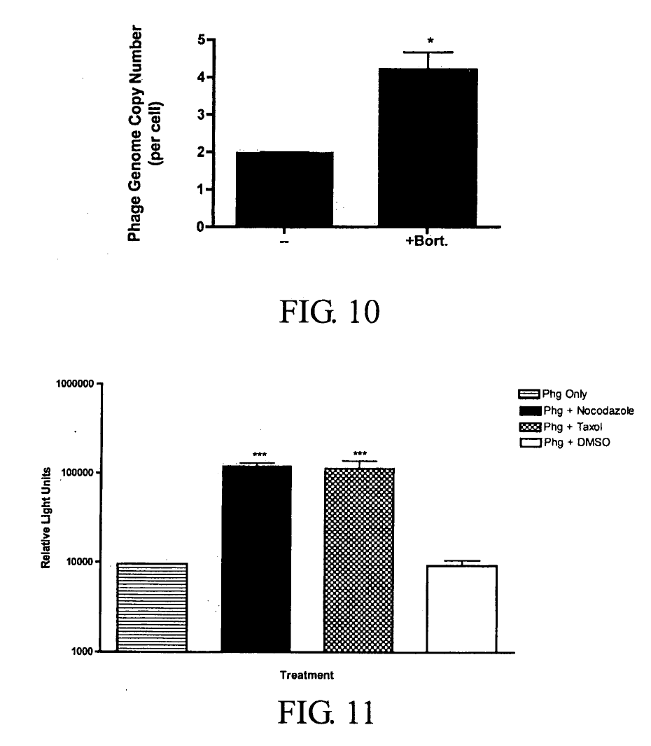

Statistical analysis. FIGS. 1 and 3-10 show results that are representative of at least three separate experiments with similar results, except for FIG. 10 (which was repeated twice). Data represent mean values of analyses performed in triplicate, and error bars denote the standard error of these means. Statistical significance was taken at p<0.05, and was calculated using one-way ANOVA with Tukey's post test, unless otherwise indicated.

Results

To analyze the function of the proteasome in phage-mediated gene transfer, HEK 293A cells and COS-7 cells were incubated with luciferase encoding phage particles, in the presence or absence of three different pharmacologic inhibitors of proteasome activity (lactacystin, bortezomib and MG132). Lactacystin is an irreversible inhibitor of the 20S-proteasome, while bortezomib and MG132 are reversible inhibitors of the 26S-proteasome complex. For these experiments, both wild-type phage particles bearing the native lambda phage coat protein (WT-gpD) as well as modified particles that displayed a PEST-like motif at high density on their surface (Tpell-gpD) were used. The latter phage were generated by producing genetically gpD-deficient lambda phage particles in E. coli host cells that expressed a recombinant derivative of gpD, fused to a truncated PEST-like motif derived from seeligeriolysin O, a cholesterol-dependent cytolysin of Listeria seeligeri.

PEST motifs are rich in proline (P), glutamic acid (D), aspartic acid (E) and serine (S) or threonine (T) residues and serve to direct proteins for proteasomal degradation (Rechsteiner and Rogers, Trends Biochem. Sci. 21(7):267-7 1, 1996). Therefore, the PEST motif used in the experiments (SPAETPESPPATPK (SEQ ID NO: 1); designated hereafter as “Tpell”) might cause phage particles bearing this element to become targeted to proteasomes. Therefore, the gene transfer efficiency in HEK 293 cells by phage particles bearing the Tpell-modified gpD coat protein was compared to that of phage particles bearing the wild-type (unmodified) coat protein, both in the presence and absence of pharmacologic inhibitors of the proteasome. The results are shown in FIGS. 1A and 1B. The efficiency of gene transfer by phage particles bearing either the wild-type or the Tpell-modified coat protein was strongly enhanced in the presence of the proteasome inhibitors (FIGS. 1A and 1B). All three proteasomal inhibitors enhanced gene transfer from wild type and Tpell modified particles in HEK 293 cells. MG132 showed had the strongest effect on phage-mediated luciferase expression (enhancing it by 5-10 fold). A similar trend was observed in cells treated with lactacystin and bortezomib.

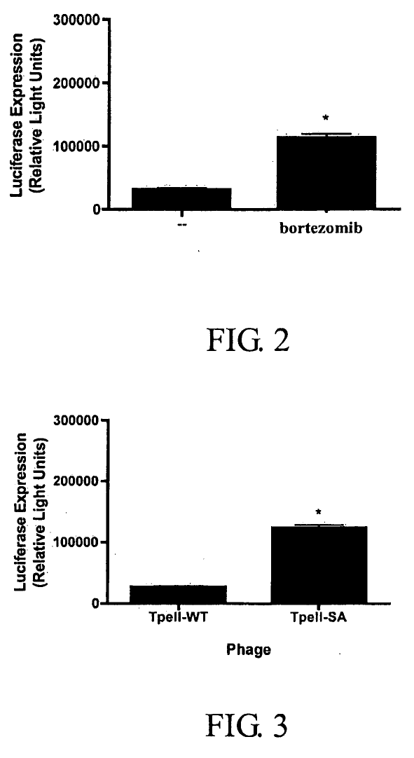

Bortezomib was found to be the least cytotoxic of the proteasome inhibitors tested and was better tolerated by HEK 293 cells than the other drugs. Therefore, it was evaluated whether extended (24 hour) exposure to bortezomib might result in improved phage-mediated gene transfer in HEK 293 cells. The results in FIG. 2 show that extended proteasome inhibition using bortezomib resulted in a robust, statistically significant, enhancement of phage-mediated gene transfer in HEK 293 cells.

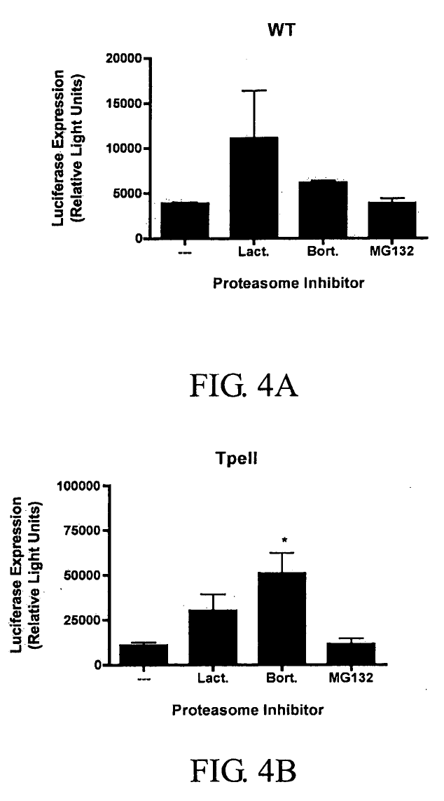

The data in FIGS. 1A, 1B, 4A and 4B show that (1) HEK 293 cells were roughly 10-fold more susceptible to phage-mediated gene transfer than COS cells, and (2) transduction with Tpell phage resulted in approximately 10-fold higher levels of luciferase expression in both 293 and COS cells, when compared to WT phage. Since proteasome inhibition lead to a robust increase in gene transfer but Tpell phage had higher levels of transduction than wild-type page, experiments were conducted to determine whether the presence of an intact PEST motif is in fact necessary for enhancement of phage-mediated gene transfer by the Tpell phage. To do this, a plasmid expression construct was developed that encoded the major lambda phage coat protein, gpD, fused to either wild-type “Tpell” (“Tpell-WT”) or to a mutated derivative of “Tpell” in which the two serine residues were substituted by alanines (“Tpell-SAM”) thereby eliminating the PEST element in Tpell. Luciferase-encoding phage particles were then generated displaying these peptides on their surface and were used to transduce HEK 293 cells. Analysis of luciferase expression in cell lysates revealed that phage-mediated gene transfer efficiency in HEK 293 cells was in fact enhanced by surface display of the mutated, non-functional PEST motif (FIG. 3). Thus, the PEST motif is not required for Tpell phage to transduce mammalian cells more efficiently than wild-type phage particles.

Next, an experiment was performed to confirm that the proteasome inhibitors enhanced phage-mediated gene transfer in other cell types. COS cells were selected for these experiments, since they have been used in previous studies on phage-mediated gene delivery (Eguchi et al., J. Biol. Chem. 276(28):26204-10, 2001). In COS cells, bortezomib and lactacystin both enhanced luciferase expression by 2-4 fold (FIGS. 4A and 4B).

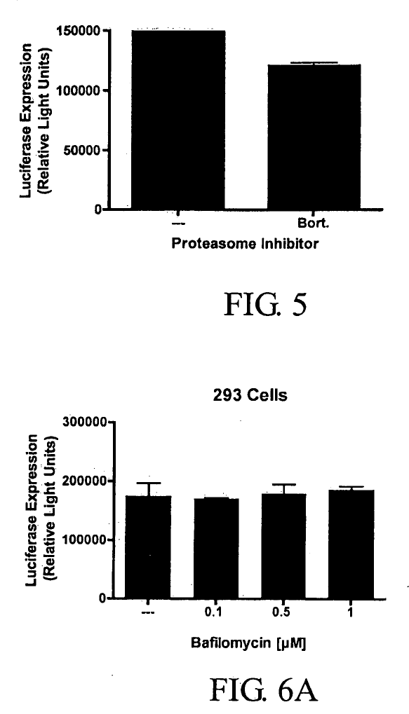

Inhibition of proteasome activity is known to prevent degradation of cellular proteins and can exert a strong effect on the activity of cellular transcription factors, such as NFκB. Therefore, a control experiment was performed to determine whether proteasome inhibition exerted an effect on luciferase expression from a non-phage based gene transfer agent containing the same luciferase expression cassette present in λD1180(luc). For this experiment, HEK 293 cells were transiently transfected with a plasmid containing the firefly luciferase reporter gene under the transcriptional control of the human CMV major immediate-early promoter (pCMV:luc); cells were then incubated in the presence or absence of bortezomib, prior to harvest and analysis of luciferase activity in cell lysates. As shown in FIG. 5, bortezomib had no effect on luciferase expression in pCMV:luc transfected HEK 293 cells. Without meaning to be limited by theory, proteasome inhibitors enhanced gene transfer efficiency by phage vectors not because of effects on gene expression/promoter activity, but rather through effects on the intracellular degradation or trafficking of phage particles.

### Example 2

### Chloroquine and Inhibition of Cathepsins Enhance Phage-Mediated Gene Transfer

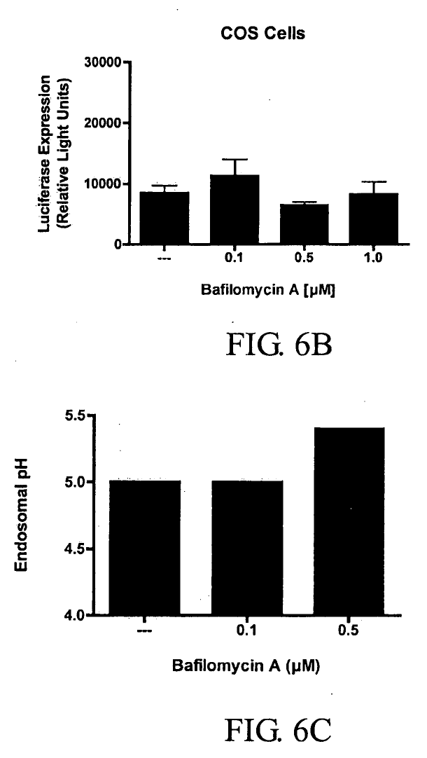

Many animal viruses enter the cell through an endocytic pathway, and infection of cells by these viruses can be strongly influenced by endosomal pH. To investigate the role of endosome inhibition in phage-mediated gene transfer, experiments were conducted using bafilomycin A1, a specific inhibitor of the vascular H+-ATPases. As shown in FIGS. 6A and 6B, bafilomycin had no effect on phage-mediated gene transfer in either HEK 293 or COS cells. To confirm that the concentrations of bafilomycin used were sufficient to raise endosomal pH, a control experiment was performed in which FITC-dextran-tetramethylrhodamine was added to HEK 293 cells in the presence or absence of bafilomycin Al, and endosomal pH was then assessed by flow cytometry. As shown in FIG. 6C, treatment of the cells with 500 nM bafilomycin A1 was sufficient to raise endosomal pH. Thus, the lack of an effect of bafilomycin A1 on phage-mediated gene transfer efficiency cannot be attributed to a failure of this agent to inhibit endosomal acidification.

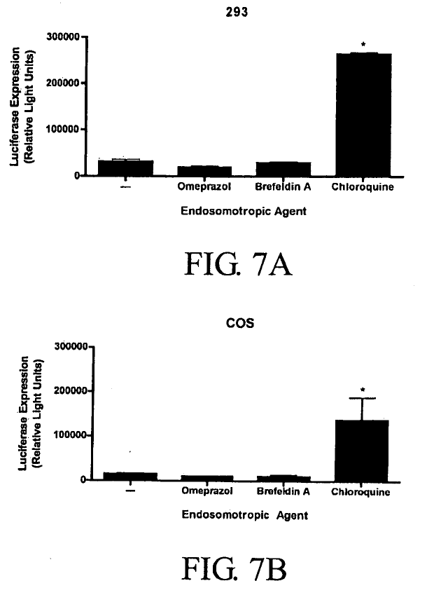

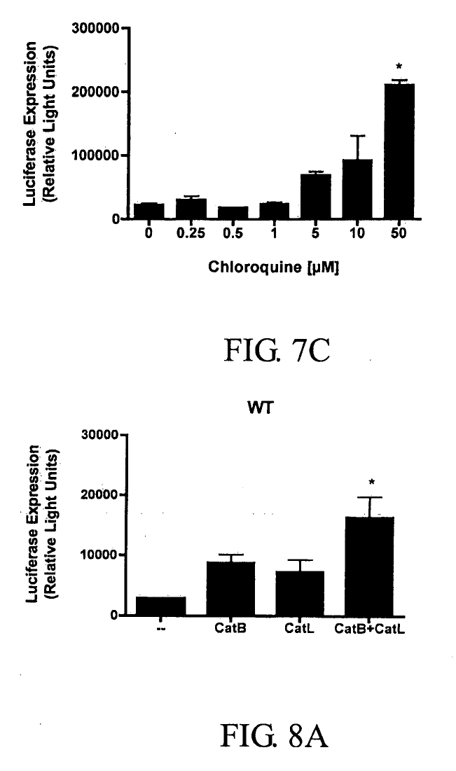

In order to confirm the results obtained with bafilomycin A1, studies were performed using additional endosomotropic agents. These included (1) omeprazole, an inhibitor of proton pump H+-K+ATPases; (2) brefeldin A, an inhibitor of early-to-late endosome transition; and (3) chloroquine, a lysosomotropic agent that accumulates in acidic endosomes to increase endosomal pH. Of these three endosomotropic drugs, only chloroquine enhanced phage-mediated gene transfer (FIGS. 7A and 7B).

Since this experiment used a high dose of chloroquine (50 or 70 μM, respectively, in HEK 293 or COS cells), the dose-response effect associated with chloroquine treatment was examined. The results of the study (FIG. 7C) showed that phage-mediated luciferase expression was significantly enhanced only when cells were incubated with a high concentration of chloroquine (50 μM). Lower concentrations of chloroquine, including concentrations that are typically sufficient to prevent endosomal acidification, had only a modest and non-statistically significant effect on phage-mediated gene transfer (FIG. 7C).

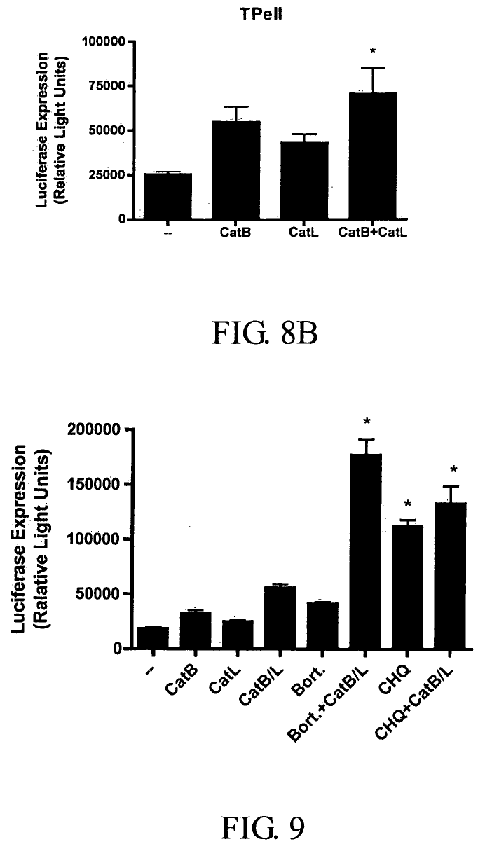

In light of these data, high concentrations of chloroquine may enhance phage-mediated gene transfer through a mechanism unrelated to the inhibition of endosome acidification. One such mechanism includes chloroquine inhibition of intracellular protein degradation and activity of cathepsin B1. Given the ability of this protease to interact with viruses in the lysosome, it was directly tested whether inhibition of lysosomal proteases enhance phage-mediated gene transfer. Specifically, it was determined whether inhibitors of cathepsin B and cathepsin L (catB and catL) promote phage gene transfer in HEK 293A cells. As shown in FIGS. 8A and 8B, inhibition of either catB or catL alone led to an increase in phage-mediated luciferase expression. Simultaneous inhibition of both cathepsins led to a robust, statistically significant increase in phage-mediated gene transfer. This observation confirms that lysosomal proteases target incoming phage particles for degradation, and thereby limit the efficiency of phage-mediated gene transfer in mammalian cells.

Experiments were performed in which cells were exposed to luciferase-encoding phage particles in the presence or absence of lysosomal protease inhibitors either alone, or in combination with (1) a proteasomal inhibitor (bortezomib) or (2) chloroquine (at a high concentration, expected to result in inhibition of lysosomal proteases). As shown in FIG. 9, this experiment revealed that the lysosomal protease inhibitors synergized with the proteasome inhibitor (bortezomib) to enhance phage-mediated luciferase expression in HEK 293 cells. This observation is consistent with the hypothesis that proteasomal inhibitors and lysosomal protease inhibitors enhance the efficiency of phage-mediated gene transfer via distinct mechanistic pathways.

In contrast, exposure of cells to a high concentration of chloroquine (CHQ) resulted in a strong and statistically significant increase in phage-mediated luciferase expression that was not substantially enhanced by co-treatment with lysosomal protease inhibitors (FIG. 9). This finding shows that chloroquine enhances phage-mediated gene transfer principally via inhibition of lysosomal proteases. Without meaning to be limited by theory, the fact that high dose chloroquine exerted a much stronger effect on phage-mediated gene transfer than inhibition of the two cathepsins (catB plus catL) is most likely a reflection of the fact that CHQ may inhibit other lysosomal proteases in addition to catB and catL.

To understand how proteasome inhibition enhanced the efficiency of phage-mediated gene transfer, HEK 293 cells were incubated with luciferase-encoding phage vector in the presence or absence of bortezomib, harvested after 24 hours, washed thoroughly to remove residual surface bound phage, and used to prepare nuclear DNA extracts. Phage genomic DNA within these extracts was then quantitated by DNA PCR analysis, and the results are presented in FIG. 10 (normalized in terms of the number of copies of nuclear phage DNA per cell). The data show that exposure of the cells to the proteasome inhibitor resulted in a statistically significant increase in the nuclear accumulation of phage DNA (p<0.01).

In summary, these data show proteasome inhibition enhanced phage-mediated gene transfer by promoting the intracellular survival of phage particles, thereby allowing a greater number of phage genomes to escape the cytoplasm, penetrate the nucleus and initiate gene expression.

### Example 3

### Treatment of Cells with a Microtubule Inhibitor Enhanced Phage-Mediated Gene Transfer

Wild-type lambda phage particles were incubated with gpD-specific rabbit IgG antibodies, to generate phage:antibody complexes. These were then added to COS cells that had been stably transfected with expression plasmids encoding a cellular Fc receptor (CD64) and its associated gamma chain. Phage were added to cells that had been pretreated for 30 minutes in the presence or absence of nocodazole (5 μM) or paclitaxel (20 μg/ml). Cells were maintained in the continuous presence of the microtubule inhibitors, harvested 48 hours later and lysed and luciferase activity was measured. Addition of nocodazole or paclitaxel resulted in a large (10-50-fold) increase in gene transfer efficiency (FIG. 11). These data indicate that microtubule inhibitors, paclitaxel and nocodazole, resulted in a large increase in phage-mediated gene transfer efficiency.

### Example 4

### Treatment of Cells with a Microtubule Inhibitor Enhanced Plasmid-Mediated Gene Transfer

A DNA plasmid encoding a luciferase reporter gene was mixed with Lipofectamine™ (Invitrogen, Carlsbad, Calif.). This was then added to COS cells that had been stably transfected expression plasmids encoding a cellular Fc receptor

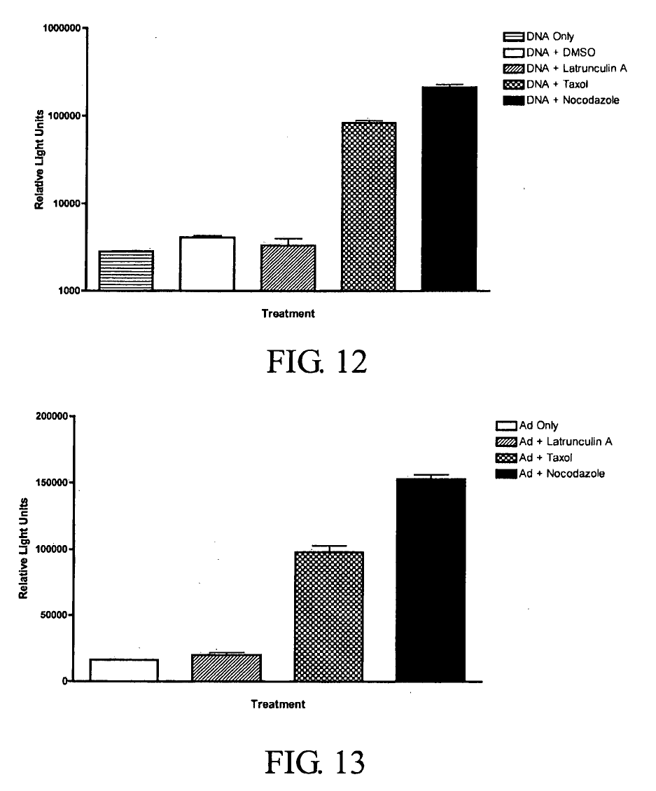

(CD64) and its associated gamma chain. DNA was added to cells that had been pretreated for 30 minutes in the presence or absence of the microtubule inhibitors, nocodazole (5 μM) or paclitaxel (20 μg/ml), or the actin polymerization inhibitor, latrunculin A (120 nM). Cells were harvested 48 hours after transfection and lysed, and luciferase activity was measured. Addition of nocodazole or paclitaxel resulted in a large increase in gene transfer efficiency (FIG. 12).

These data indicate that microtubule inhibitors, paclitaxel and nocodazole, resulted in a large (approx 50-fold) increase in gene transfer efficiency. In contrast, addition of DMSO or latrunculin A had no effect on the efficiency of plasmid DNA expression.

### Example 5

### Treatment of Cells with a Microtubule Inhibitor Enhanced Viral Vector-Mediated Gene Transfer

Latrunculin A (120 nM), paclitaxel (Taxol® (Bristol Meyers Squibb, Princeton, N.J.), 20 μg/ml), or nocodazole (5 μM) was added to cells 30 minutes prior to transduction of COS-7 cells with a luciferase-expressing adenovirus vector (AdLucGFP) at a multiplicity of infection (MOI) of 10. Media were changed 24-hours post-transfection, and cells were lysed in Passive Lysis Buffer 24 hours later. Protein quantities were standardized and luciferase activity was measured in the cell lysates.

These data indicate that microtubule inhibitors, paclitaxel and nocodazole, resulted in a substantial (approx 4-5 fold) increase in gene transfer efficiency (FIG. 13). In contrast, addition of DMSO or latrunculin A had no effect on the efficiency of plasmid DNA expression.

1. A method of increasing expression of an exogenous gene in a cell, comprising:
a) contacting the cell with a bacteriophage, plasmid or viral vector comprising the exogenous gene; and
b) contacting the cell with one or more agents selected from the group consisting of a proteasome inhibitor, a lysosomal inhibitor and a microtubule inhibitor.

2. The method of claim 1, wherein contacting the cell with the agent results in an increase in expression of the exogenous gene in the cell as compared to a control.

3. A method of delivering an antigen delivery vector to a cell, comprising:
a) contacting the cell with an antigen delivery vector encoding an antigen, wherein the antigen delivery vector is a bacteriophage, plasmid or viral vector; and
b) contacting the cell with one or more agents selected from the group consisting of a proteasome inhibitor, a lysosomal inhibitor and a microtubule inhibitor.

4. The method of claim 1, wherein the cell is in vitro.

5. The method of claim 1, wherein the cell is in vivo.

6. (canceled)

7. (canceled)

8. (canceled)

9. The method of claim 1, wherein the cell is contacted with a bacteriophage and wherein the agent is a proteasome inhibitor or a lysosomal protease inhibitor or both.

10. The method of claim 1, wherein the cell is contacted with a viral vector and wherein the agent is a proteasome inhibitor, microtubule inhibitor, or a lysosomal protease inhibitor or a combination thereof.

11. (canceled)

12. The method of claim 3, wherein the antigen delivery vector is a bacteriophage and wherein the agent is a proteasome inhibitor or a lysosomal protease inhibitor or both.

13. (canceled)

14. The method of claim 3, wherein the antigen delivery vector is a viral vector and wherein the agent is a a proteasome inhibitor, microtubule inhibitor, or a lysosomal protease inhibitor or a combination thereof.

15. (canceled)

16. The method of claim 1, wherein the cell is contacted with a plasmid and wherein the agent is a microtubule inhibitor.

17. The method of claim 3, wherein the antigen delivery vector is a plasmid and wherein the agent is a microtubule inhibitor.

18. The method of claim 12, wherein the bacteriophage is a bacteriophage lambda.

19. The method of claim 9, wherein the bacteriophage is bacteriophage lambda.

20. The method of claim 10, wherein the bacteriophage lambda is modified to display PEST-like motifs on the surface of the bacteriophage.

21. The method of claim 11, wherein the bacteriophage lambda is modified to display PEST-like motifs on the surface of the bacteriophage.

22. The method of claim 1, wherein the agent is a proteasome inhibitor and wherein the proteasome inhibitor is selected from one or more of the group consisting of bortezomib, lactacystin, MG132, peptide aldehydes, epoxomicin and derivatives of epigallocatechin-3-gallate.

23. The method of claim 1, wherein the agent is a lysosomal protease inhibitor and wherein the lysosomal protease inhibitor is selected from one or more of the group consisting of a cathepsin B inhibitor, a cathepsin L inhibitor, chloroquine, antipain hydrochloride, chymostatin, trans-eposycuccinyl-1-leucylamido-(4-guanidino)butane, leupeptin, pepstatin A, CA074Me, ZPAD, serpinB3, and cystatin C.

24. The method of claim 1, wherein the agent is a microtubule inhibitor and wherein the microtubule inhibitor is selected from one or more of the group consisting of nocadozole, paclitaxel, vinblastine, vincristine, colchicine, vinorelbine, vindesine, docetaxel, ixabepilone, SB-715992, SB-743921, tryprostatin A, dolastatin 15, podophyllotoxin and rhizoxin.

25. An antigen delivery system comprising
(a) an agent selected from the group consisting of a proteasome inhibitor, a lysosomal protease inhibitor and a microtubule inhibitor; and
(b) an antigen delivery vector encoding an antigen.

26. The antigen delivery system of claim 17, wherein the antigen delivery vector is a bacteriophage and wherein the agent is a proteasome inhibitor or a lysosomal inhibitor.

27. The antigen delivery system of claim 18, wherein the bacteriophage is bacteriophage lambda.

28. The antigen delivery system of claim 19, wherein the bacteriophage lambda is modified to display PEST-like motifs on the surface of the bacteriophage.

29. The antigen delivery system of claim 17, wherein the antigen delivery vector is a viral vector and wherein the agent is a proteasome inhibitor or a lysosomal inhibitor.

30. The antigen delivery system of claim 17, wherein the antigen delivery vector is a plasmid and wherein the agent is a microtubule inhibitor.

31. The antigen delivery system of claim 17, wherein the agent is a proteasome inhibitor and wherein the proteasome inhibitor is selected from one or more of the group consisting of bortezomib, lactacystin, MG132, peptide aldehydes, epoxomicin and derivatives of epigallocatechin-3-gallate.

32. The antigen delivery system of claim 17, wherein the agent is a lysosomal protease inhibitor and wherein the lysosomal protease inhibitor is selected from one or more of the group consisting of a cathepsin B inhibitor, cathepsin L inhibitor, chloroquine, antipain hydrochloride, chymostatin, trans-eposycuccinyl-1-leucylamido-(4-guanidino)butane, leupeptin, pepstatin A, CA074Me, ZPAD, serpinB3, and cystatin C.

33. The antigen delivery system of claim 17, wherein the agent is a microtubule inhibitor and wherein the microtubule inhibitor is selected from one or more of the group consisting of nocadozole, paclitaxel, vinblastine, vincristine, colchicine, vinorelbine, vindesine, docetaxel, ixabepilone, SB-715992, SB-743921, tryprostatin A, dolastatin 15, podophyllotoxin and rhizoxin.

34. A composition comprising the antigen delivery system of any claim 25 and a pharmaceutically acceptable carrier.

35. A kit comprising
(a) an antigen delivery vector; and
(b) an agent selected from the group consisting of a proteasome inhibitor, a lysosomal protease inhibitor and a microtubule inhibitor.

36. The kit of claim 27, wherein the antigen delivery vector is a bacteriophage and wherein the agent is a proteasome inhibitor or a lysosomal protease inhibitor.

37. The kit of claim 28, wherein the bacteriophage is bacteriophage lambda.

38. The kit of claim 29, wherein the bacteriophage lambda is modified to display PEST-like motifs on the surface of the bacteriophage.

39. The kit of claim 27, wherein the antigen delivery vector is a viral vector and wherein the agent is a proteasome inhibitor or a lysosomal protease inhibitor.

40. The kit of claim 27, wherein the antigen delivery vector is a plasmid and wherein the agent is a microtubule inhibitor.

41. The kit of claim 27, wherein the agent is a proteasome inhibitor and wherein the proteasome inhibitor is selected from one or more of the group consisting of bortezomib, lactacystin, MG132, peptide aldehydes, epoxomicin and derivatives of epigallocatechin-3 -gallate.

42. The kit of claim 27, wherein the agent is a lysosomal protease inhibitor and wherein the lysosomal protease inhibitor is selected from one or more of the group consisting of a cathepsin B inhibitor, cathepsin L inhibitor, chloroquine, antipain hydrochloride, chymostatin, trans-eposycuccinyl-1-leucylamido-(4-guanidino)butane, leupeptin, pepstatin A, CA074Me, ZPAD, serpinB3, and cystatin C.

43. The kit of claim 27, wherein the agent is a microtubule inhibitor and wherein the microtubule inhibitor is selected from one or more of the group consisting of nocadozole, paclitaxel, vinblastine, vincristine, colchicine, vinorelbine, vindesine, docetaxel, ixabepilone, SB-715992, SB-743921, tryprostatin A, dolastatin 15, podophyllotoxin and rhizoxin.

44.-46. (canceled)

47. The method of claim 3, wherein the cell is in vitro.

48. The method of claim 3, wherein the cell is in vivo.

49. The method of claim 3, wherein the agent is a proteasome inhibitor and wherein the proteasome inhibitor is selected from one or more of the group consisting of bortezomib, lactacystin, MG132, peptide aldehydes, epoxomicin and derivatives of epigallocatechin-3 -gallate.

50. The method of claim 3, wherein the agent is a lysosomal protease inhibitor and wherein the lysosomal protease inhibitor is selected from one or more of the group consisting of a cathepsin B inhibitor, a cathepsin L inhibitor, chloroquine, antipain hydrochloride, chymostatin, trans-eposycuccinyl-1-leucylamido-(4-guanidino)butane, leupeptin, pepstatin A, CA074Me, ZPAD, serpinB3, and cystatin C.

51. The method of claim 3, wherein the agent is a microtubule inhibitor and wherein the microtubule inhibitor is selected from one or more of the group consisting of nocadozole, paclitaxel, vinblastine, vincristine, colchicine, vinorelbine, vindesine, docetaxel, ixabepilone, SB-715992, SB-743921, tryprostatin A, dolastatin 15, podophyllotoxin and rhizoxin.
